(19) United States
(12) Patent Application Publication
(10) Pub. No.: US 2023/0401343 A1
(43) Pub. Date: Dec. 14, 2023

(54) Three-Dimensional Modeling Method for Thermal Runaway of Lithium-Ion Battery under Different State of Charge Conditions Based on Differential Scanning Calorimeter Experiment

(71) Applicant: NANJING TECH UNIVERSITY, Nanjing (CN)

(72) Inventors: ZHIRONG WANG, Nanjing (CN); TENGFEI HE, Nanjing (CN); YANGYAN ZHENG, Nanjing (CN)

(21) Appl. No.: 18/203,291

(22) Filed: May 30, 2023

(30) Foreign Application Priority Data
Jun. 8, 2022 (CN) 202210648270.8

(51) Int. Cl. G06F 30/10; G06F 30/27

(52) U.S. Cl. CPC G06F 30/10; G06F 30/27; G06F 2111/06

(57) ABSTRACT

The present invention discloses a three-dimensional modeling method for thermal runaway of a lithium-ion battery under different state of charge (SOC) conditions based on a differential scanning calorimeter (DSC) experiment, comprising S1: obtaining an active material of a battery, and performing a DSC experiment to obtain a heat flow curve; S2: dividing the heat flow curve of the battery into a plurality of reaction peaks to obtain a reaction enthalpy of each peak of the battery; S3: analyzing the heat flow curve by utilizing a Kissinger equation to obtain activation energy and a pre-exponential factor; S4: fitting the heat flow curve of the material of the battery by using a genetic algorithm to obtain a reaction order of the active material of the lithium-ion battery; S5: establishing a thermal runaway model of the battery, and comparing simulation experimental results to verify the feasibility of the model; and S6: changing the SOC of the lithium-ion battery, and studying the influence of different SOC on the thermal runaway of the lithium-ion battery. The thermal runaway model established based on the DSC experiment according to the present invention can actually reproduce the thermal runaway reaction of the lithium-ion battery during the thermal runaway process and improve the accuracy of the model.

### TECHNICAL FIELD

The present invention belongs to the technical field of lithium-ion battery safety, and particularly relates to a three-dimensional modeling method for thermal runaway of a lithium-ion battery under different state of charge conditions based on a differential scanning calorimeter experiment.

### BACKGROUND ART

At present, due to the advantages of relatively high energy density, long life, no memory effect, relatively low self-discharge rate and the like, lithium-ion batteries are widely used in the fields such as electric vehicles, portable mobile devices, and aerospace. However, the lithium-ion batteries still have serious safety problems. The most common of these is thermal runaway caused by thermal runaway of the lithium-ion batteries.

The thermal runaway of the lithium-ion batteries can be caused by thermal, electrical, and mechanical abuse, and is a complex exothermic process involving a series of chemical reactions that release a large amount of heat and may lead to smoking, combustion, and explosion.

Adiabatic accelerating rate calorimetry (ARC) and differential scanning calorimetry (DSC) are commonly used thermal safety methods to study a chain chemical reaction process during the temperature rise of the lithium-ion battery. An effective thermal runaway model for the lithium-ion battery can be established to understand the chain chemical reaction and thermal behavior of the lithium-ion battery during the thermal runaway process, and can also be used for studying cooling methods for preventing the thermal runaway of the lithium-ion battery.

Commonly used instruments for acquiring thermodynamic parameters of the lithium-ion battery include a differential scanning calorimeter (DSC), an accelerating rate calorimeter (ARC), a C80 calorimeter and a CONE calorimeter. A thermal runaway model using the ARC, the C80 calorimeter or the CONE calorimeter for acquiring the thermodynamic parameters of the battery during the thermal runaway process can well reflect the temperature change of the battery during the thermal runaway process, but the reaction sequence and the kinetics of internal materials of the battery during the thermal runaway process are not considered. Therefore, it is not possible to analyze the heat generation proportion of each component of the battery, while the understanding of the heat generation proportion of the material of the battery also contributes to the understanding of the mechanism of thermal runaway during the thermal runaway process.

However, thermal runaway models established based on reaction kinetics in the prior art are mostly based on literature data, such as Chinese patent, titled a modeling method for thermal runaway of lithium-ion battery, with application number: 202110570368.1. The model parameters in the solution of this patent application mainly come from the parameters in the literature, while there are some differences between the materials in the literature and the thermal runaway reaction of the battery used for modeling, which will lead to relatively large error and low accuracy of the model to a large extent.

### SUMMARY OF THE INVENTION

It is an object of the present invention to provide a three-dimensional modeling method for thermal runaway of a lithium-ion battery under different state of charge conditions based on a differential scanning calorimeter experiment, so as to solve the technical problems of relatively large error and low accuracy of a thermal runaway model for a lithium-ion battery established based on reaction kinetics in the prior art.

In order to solve the above technical problems, the present invention is implemented by adopting the following technical solution:

the three-dimensional modeling method for thermal runaway of a lithium-ion battery under different state of charge conditions based on a differential scanning calorimeter experiment comprises the following steps:

S1: acquiring an active material of the lithium-ion battery with a set charge value, and performing a differential scanning calorimeter (DSC) experiment on the active material to acquire heat flow curves of the active material of the lithium-ion battery at different temperature rise rates respectively;

S2: dividing the heat flow curve of the battery into a plurality of reaction peaks by using a non-linear fitting method to obtain a reaction enthalpy of each peak of the battery;

S3: analyzing the heat flow curves acquired in step S1 by utilizing a Kissinger equation to obtain activation energy and a pre-exponential factor of the active material of the lithium-ion battery;

S4: fitting the heat flow curves of the material of the battery by using a genetic algorithm to obtain a reaction order of the active material of the lithium-ion battery;

S5: establishing a thermal runaway model for the lithium-ion battery, and taking parameters of the active material of the lithium-ion battery obtained in steps S2-S4 into the model to acquire a simulated result of the thermal runaway of the lithium-ion battery, and comparing the simulated result with an experimental result of actual thermal runaway of the lithium-ion battery in step S1 to verify the feasibility of the model; and

S6: changing a state of charge of the lithium-ion battery, respectively repeating steps S1-S5 under different states of charge, and studying the influence of different states of charge on the thermal runaway of the lithium-ion battery.

In the present invention, the thermal runaway model is established by performing the DSC experiment on the lithium-ion battery to acquire kinetic parameters, so that the actual situation of the thermal reaction process of the battery can be reflected, and the influence of different SOC on the thermal runaway of the battery can be analyzed by performing experiments on the battery under SOC conditions. The simulation method is high in accuracy, saves the cost of thermal runaway experiments, and the like.

For further optimization, the step S1 comprises the following steps:

S11: charging the lithium-ion battery to a set electric quantity value by utilizing a charge-discharge meter, and then placing the lithium-ion battery into a glove box for disassembly to acquire a cathode active material, a anode active material, an electrolyte and a separator of the battery, and making the cathode active material, the anode active material and the separator into powder; and

mixing the cathode active material with the anode active material in an equal proportion according to positive and anode active materials in a total battery, and denoting as A; and mixing the anode active material with the electrolyte in the equal proportion, and denoting as B; denoting the cathode active material as C; denoting the separator as D; denoting the electrolyte as E; mixing a cathode with the electrolyte in the equal proportion, and denoting as F; and denoting a anode as G; and

S12: placing the four substances A, B, C and D in step S11 into a DSC apparatus by utilizing a standard aluminium crucible respectively, and performing experiments at four temperature rise rates of 10° C. min⁻¹, 15° C. min⁻¹, 20° C. min⁻¹ and 25° C. min⁻¹ respectively;

For further optimization, in the step S3, activation energy and pre-exponential factors of different reaction peaks are respectively obtained by using Kissinger's equation fitting based on reaction peak temperatures at different temperature rise rates and reaction enthalpies of different peaks, wherein Kissinger's equation is as follows:

$$ln ( \frac{{α}_{i}}{{T}_{i}^{2}} ) = ln ( \frac{{A}_{x}}{{E}_{a , x}} ) - \frac{{E}_{a , x}}{{RT}_{i}} , i = 1 , 2 , 3 … u ;$$

in the above formula, R is an ideal gas state constant, 8.314 J/mol/K; Aₓ is the pre-exponential factor of the material of the battery; E_{a,x} is the activation energy of the material of the battery; Ti is the peak temperature; u is a serial number of the changing temperature rise rate; and α is the temperature rise rate.

$$ln ( \frac{{α}_{i}}{{T}_{i}^{2}} )$$

is taken as a dependent variable,

$$\frac{1}{{T}_{i}}$$

is taken as an independent variable, linear fitting is performed, the obtained slope of a straight line is multiplied by R to obtain the activation energy E_{a,x} of the reaction peak, while the reaction pre-exponential factor of the reaction peak can be obtained by an intercept

$$ln ( \frac{{A}_{x}}{{E}_{a , x}} )$$

of the straight line.

For further optimization, in the step S4, in order to fit the heat flow curves of the material of the battery by using the genetic algorithm to obtain the reaction order of the material of the battery, a heat generation formula of the lithium-ion battery used is as follows:

$${Qm}_{x} = Δ {H}_{x} \cdot {K}_{x} \cdot m ;$$

$${K}_{x} = {A}_{x} \cdot exp ( - \frac{{E}_{a , x}}{RT} ) \cdot f ( {c}_{x} ) ;$$

$$\frac{{dc}_{x}}{dt} = - {K}_{x} , {c}_{x , 0} = 1 ;$$

$$f ( {c}_{x} ) = d \cdot [ {( 1 - {c}_{x} )}^{a} + p ] \cdot ( {c}_{x}^{b} ) ;$$

in the above formula, Kₓ is a decomposition reaction rate of the material of the battery, 1/s; cₓ is a reaction concentration of the material of the battery; ΔHₓ is the reaction enthalpy of the material of the battery, J/g; Qmₓ is heat generated by the material of the battery, W; c_{x,0} is an initial value of the reaction concentration of the material of the battery; a, b are reaction orders; p, d are reaction orders; T is a temperature of the material of the battery, K; and m is a mass of the material of the battery, and is normalized to 1 mg in this formula;

a peak temperature and the reaction enthalpy of the battery can be obtained by step S2; and the pre-exponential factor and activation energy of the material of the battery can be obtained by step S3. Therefore, since values of the above parameters except a, b, p, d are all known, an objective function is set as Qmₓ and variables are set as a, b, p, d, the above formula is put into an MATLAB program, and a Qmₓ value of the best fitting objective function is obtained by utilizing the genetic algorithm to obtain a, b, p, d values best matched with the Qmₓ value.

For further optimization, in the step S5, the three-dimensional model is established according to actual sizes of the lithium-ion battery, and the model comprises a three-dimensional thermal runaway heat generation model and a three-dimensional thermal runaway heat conduction model; and

the three-dimensional thermal runaway heat generation model is established by calculating heat generated by the battery during a thermal runaway process based on the DSC experimental heat of the lithium-ion battery; in a thermal runaway experiment, as the temperature rises, the electrolyte of the lithium-ion battery can react with the anode, further the separator is melted, the cathode and the anode are in direct contact, and then the cathode and the anode can react with each other; as the temperature further rises, the cathode is decomposed and releases heat; and heat transfer is performed inside the battery by means of heat conduction, and convection heat transfer and heat radiation heat transfer are performed between a surface of the battery and an environment.

For further optimization, in the step S5, the established model is verified through an experimental method, and the method specifically comprises the following steps:

S51: placing the lithium-ion battery into an adiabatic accelerating rate calorimeter to perform the thermal runaway experiment; and

S52: measuring a temperature change of the lithium-ion battery during the thermal runaway process by using a thermocouple, and comparing a measured result with a model result.

For further optimization, in the step S6, the battery is charged to different states of charge (SOC), namely 100% SOC, 80% SOC, 60% SOC, 40% SOC and 20% SOC respectively, by the charge-discharge meter.

Compared with the prior art, the present invention has the following beneficial effects:

1. The thermal runaway model established based on the DSC experiment according to the present invention can actually reproduce the thermal runaway reaction of the lithium-ion battery during the thermal runaway process and improve the accuracy of the model.

2. In the present invention, the thermal runaway model for the battery can be obtained by testing only a small number of batteries, and a thermal runaway propagation model can be established by utilizing the model to study suppression methods for the thermal runaway of the lithium-ion battery.

3. The heat generation ratio of positive and anode active materials of the battery during the thermal runaway process can be actually calculated by the model, and then parts with relatively high heat generation can be modified to reduce the risk of the thermal runaway of the battery.

4. The thermal runaway process of the lithium-ion battery under different SOC conditions can be accurately simulated by the model.

### BRIEF DESCRIPTION OF THE DRAWINGS

FIG. 1 is a flowchart of a three-dimensional modeling method for thermal runaway of a lithium-ion battery under different state of charge conditions based on a differential scanning calorimeter experiment according to the present invention;

FIG. 2 shows appearance and sizes of a battery used for modeling in example 1 of the present invention;

FIG. 3 is a comparison diagram of heat flow curves of a material of the battery under 20° C./min temperature rise conditions and 100% SOC conditions; wherein FIG. 3(a) is a comparison diagram of heat flow curves of a cathode, the cathode+a anode, and the anode of the battery; FIG. 3(b) is a comparison diagram of heat flow curves of the anode, an electrolyte+the anode, and the anode of the battery; and FIG. 3(c) is a comparison diagram of heat flow curves of the cathode, the cathode+the electrolyte, and the electrolyte of the battery;

FIG. 4 shows non-linear fitting results of heat flow curves of the cathode+the anode, the anode+the electrolyte of the lithium-ion battery under 100% SOC conditions and reaction enthalpies of the cathode; wherein FIG. 4(a) shows the non-linear fitting result of the cathode+the anode of the lithium-ion battery under 100% SOC conditions; FIG. 4(b) shows the non-linear fitting result of the anode+the electrolyte of the lithium-ion battery under 100% SOC conditions; and FIG. 4(c) shows the reaction enthalpies of two reaction peaks of the cathode of the lithium-ion battery under 100% SOC conditions;

FIG. 5 is a comparison diagram of fitting and experimental results corresponding to each substance under different temperature rise rate conditions and 100% SOC conditions in DSC experiments; wherein FIG. 5(a) shows the fitting and experimental results of the cathode+the anode under different temperature rise rate conditions and 100% SOC conditions in the DSC experiments; FIG. 5(b) shows the fitting and experimental results of the anode+the electrolyte under different temperature rise rate conditions and 100% SOC conditions in the DSC experiments; FIG. 5(c) shows the fitting and experimental results of the cathode under different temperature rise rate conditions and 100% SOC conditions in the DSC experiments; and FIG. 5(d) shows the fitting and experimental results of a separator under different temperature rise rate conditions and 100% SOC conditions in the DSC experiments;

FIG. 6 is a comparison diagram of fitting and experimental results corresponding to each substance under different temperature rise rate conditions and 80% SOC conditions in experiments; wherein FIG. 6(a) shows the fitting and experimental results of the cathode+the anode under different temperature rise rate conditions and 80% SOC conditions in the DSC experiments; FIG. 6(b) shows the fitting and experimental results of the anode+the electrolyte under different temperature rise rate conditions and 80% SOC conditions in the DSC experiments; and FIG. 6(c) shows the fitting and experimental results of the cathode under different temperature rise rate conditions and 80% SOC conditions in the DSC experiments;

FIG. 7 is a comparison diagram of fitting and experimental results corresponding to each substance under different temperature rise rate conditions and 60% SOC conditions in experiments; wherein FIG. 7(a) shows the fitting and experimental results of the cathode+the anode under different temperature rise rate conditions and 60% SOC conditions in the DSC experiments; FIG. 7(b) shows the fitting and experimental results of the anode+the electrolyte under different temperature rise rate conditions and 60% SOC conditions in the DSC experiments; and FIG. 7(c) shows the fitting and experimental results of the cathode under different temperature rise rate conditions and 60% SOC conditions in the DSC experiments;

FIG. 8 is a comparison diagram of fitting and experimental results corresponding to each substance under different temperature rise rate conditions and 40% SOC conditions in experiments; wherein FIG. 8(a) shows the fitting and experimental results of the cathode+the anode under different temperature rise rate conditions and 40% SOC conditions in the DSC experiments; FIG. 8(b) shows the fitting and experimental results of the anode+the electrolyte under different temperature rise rate conditions and 40% SOC conditions in the DSC experiments; and FIG. 8(c) shows the fitting and experimental results of the cathode under different temperature rise rate conditions and 40% SOC conditions in the DSC experiments;

FIG. 9 is a comparison diagram of fitting and experimental results corresponding to each substance under different temperature rise rate conditions and 20% SOC conditions in experiments; wherein FIG. 9(a) shows the fitting and experimental results of the cathode+the anode under different temperature rise rate conditions and 20% SOC conditions in the DSC experiments; FIG. 9(b) shows the fitting and experimental results of the anode+the electrolyte under different temperature rise rate conditions and 20% SOC conditions in the DSC experiments; and FIG. 9(c) shows the fitting and experimental results of the cathode under different temperature rise rate conditions and 20% SOC conditions in the DSC experiments;

FIG. 10 is a comparison diagram of experimental results and simulated results under different SOC conditions; wherein FIG. 10(a) is a comparison diagram of the experimental result and the simulated result under 100% SOC; FIG. 10(b) is a comparison diagram of the experimental result and the simulated result under 80% SOC; FIG. 10(c) is a comparison diagram of the experimental result and the simulated result under 60% SOC; FIG. 10(d) is a comparison diagram of the experimental result and the simulated result under 40% SOC; and FIG. 10(e) is a comparison diagram of the experimental result and the simulated result under 20% SOC; and

FIG. 11 shows diagrams of four different heat generation ratios and changes of heat generation with SOC in simulated results in example 1 according to the present invention; FIG. 11(a) is a diagram showing the heat generation proportion under 100% SOC; FIG. 11(b) is a diagram showing the heat generation proportion under 80% SOC; FIG. 11(c) is a diagram showing the heat generation proportion under 60% SOC; FIG. 11(d) is a diagram showing the heat generation proportion under 40% SOC; FIG. 11(e) is a diagram showing the heat generation proportion under 20% SOC; and FIG. 11(f) is a diagram showing the heat generation trend of each part under different SOC.

### DETAILED DESCRIPTION OF THE INVENTION

The specific embodiments of the present invention are given below, and the technical solution of the present invention is clearly and completely described in combination with the accompanying drawings. Obviously, the examples described are only partial examples of the present invention, but not all examples. Based on the examples in the present invention, all other examples obtained by a person of ordinary skill in the art without making creative labor are within the protection scope of the present invention.

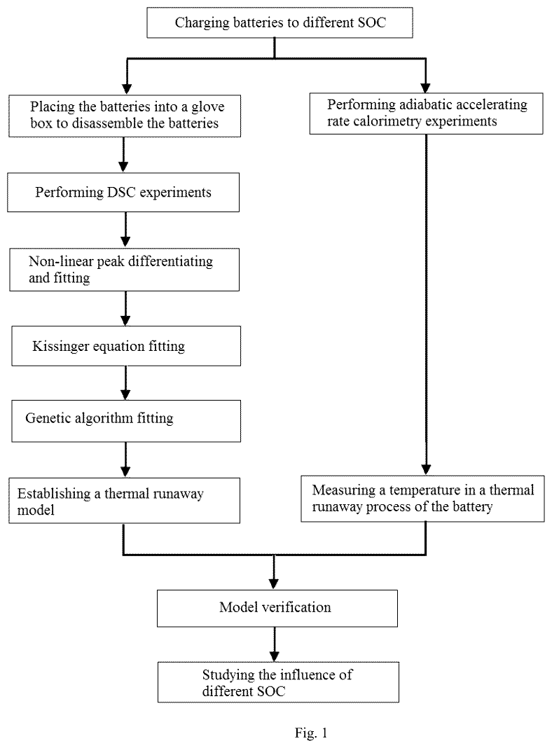

As shown in FIG. 1, a three-dimensional modeling method for thermal runaway of a lithium-ion battery under different state of charge (SOC) conditions based on a differential scanning calorimeter (DSC) experiment comprises the following steps:

S1: an active material of the lithium-ion battery with a set charge value is acquired, and the DSC experiment is performed on the active material to acquire heat flow curves of the active material of the lithium-ion battery at different temperature rise rates respectively;

S11: the lithium-ion battery is charged to a set electric quantity value by utilizing a charge-discharge meter, and then the lithium-ion battery is placed into a glove box for disassembly to acquire a cathode active material, a anode active material, an electrolyte and a separator of the battery, and the cathode active material, the anode active material and the separator are made into powder; and

a part of the cathode active material is mixed with the anode active material, and denoted as A; and a part of the anode active material is mixed with the electrolyte and the separator, and denoted as B; the cathode active material is denoted as C; and the separator is denoted as D;

S12: the four substances A, B, C and D in step S11 are placed into a DSC apparatus by utilizing a standard aluminium crucible respectively, and experiments are performed at four temperature rise rates of 10° C. min⁻¹, 15° C. min⁻¹, 20° C. min⁻¹ and 25° C. min⁻¹ respectively;

S2: the heat flow curve of the battery is divided into a plurality of reaction peaks by using a non-linear fitting method to obtain a reaction enthalpy of each peak of the battery;

S3: activation energy and pre-exponential factors of different reaction peaks are respectively obtained by using Kissinger's equation fitting based on reaction peak temperatures at different temperature rise rates and reaction enthalpies of different peaks, wherein Kissinger's equation is as follows:

$$ln ( \frac{{α}_{i}}{{T}_{i}^{2}} ) = ln ( \frac{{A}_{x}}{{E}_{a , x}} ) - \frac{{E}_{a , x}}{{RT}_{i}} , i = 1 , 2 , 3 ... u ;$$

in the above formula, R is an ideal gas state constant, 8.314 (J/mol/K); Aₓ is the pre-exponential factor of the material of the battery; E_{a,x} is the activation energy of the material of the battery; Tᵢ is the peak temperature; u is a serial number of the changing temperature rise rate; and α is the temperature rise rate;

$$ln ( \frac{{α}_{i}}{{T}_{i}^{2}} )$$

is taken as a dependent variable,

$$\frac{1}{{T}_{i}}$$

is taken as an independent variable, linear fitting is performed, the obtained slope of a straight line is multiplied by R to obtain the activation energy E_{a,x} of the reaction peak, while the reaction pre-exponential factor of the reaction peak can be obtained by an intercept

$$ln ( \frac{{A}_{x}}{{E}_{a , x}} )$$

of the straight line;

S4: the heat flow curves of the material of the battery are fitted by using a genetic algorithm to obtain a reaction order and a constant of the active material of the lithium-ion battery, wherein a heat generation formula of the lithium-ion battery used is as follows:

$${Qm}_{x} = Δ {H}_{x} \cdot {K}_{x} \cdot m ;$$

$${K}_{x} = {A}_{x} \cdot exp ( - \frac{{E}_{a , x}}{RT} ) \cdot f ( {c}_{x} ) ;$$

$$\frac{{dc}_{x}}{dt} = - {K}_{x} , {c}_{x , 0} = 1 ;$$

$$f ( {c}_{x} ) = d \cdot [ {( 1 - {c}_{x} )}^{a} + p ] \cdot ( {c}_{x}^{b} ) ;$$

in the above formula, Kₓ is a decomposition reaction rate of the material of the battery, 1/s; cₓ is a reaction concentration of the material of the battery; ΔHₓ is the reaction enthalpy of the material of the battery, J/g; Qmₓ is heat generated by the material of the battery, W; c_{x,0} is an initial value of the reaction concentration of the material of the battery; a, b are reaction orders; p, d are reaction orders; T is a temperature of the material of the battery, K; and m is a mass of the material of the battery, and is normalized to 1 mg in this formula;

a peak temperature and the reaction enthalpy of the battery can be obtained by step S2; the pre-exponential factor and activation energy of the material of the battery can be obtained by step S3; therefore, since values of the above parameters except a, b, p, d are all known, an objective function is set as Qmₓ and variables are set as a, b, p, d, the above formula is put into an MATLAB program, and a Qmₓ value of the best fitting objective function is obtained by utilizing the genetic algorithm to obtain a, b, p, d values best matched with the Qmₓ value;

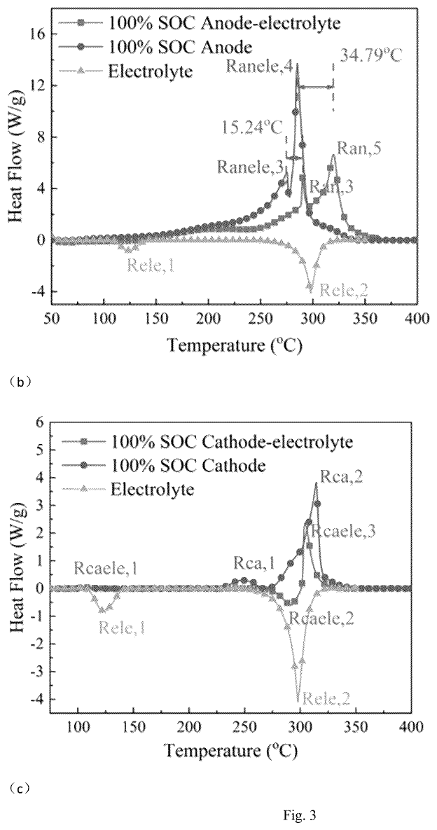

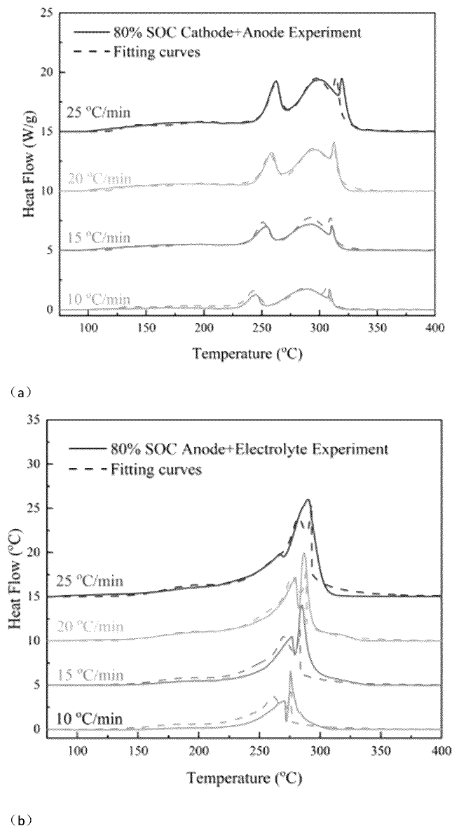

S5: a thermal runaway model for the lithium-ion battery is established, the three-dimensional model is established according to actual sizes of the lithium-ion battery, and the model comprises a three-dimensional thermal runaway heat generation model and a three-dimensional thermal runaway heat conduction model; the three-dimensional thermal runaway heat generation model is established by calculating heat generated by the battery in a thermal runaway process based on the DSC experimental heat of the lithium-ion battery; it can be obtained from the heat flow curves of the material of the battery in FIG. 3 and FIG. 5(d), in a thermal runaway experiment, as the temperature rises, the electrolyte of the lithium-ion battery can react with the anode, further the separator is melted, the cathode and the anode are in direct contact, and then the cathode and the anode can react with each other; as the temperature further rises, the cathode is decomposed and releases heat; and heat transfer is performed inside the battery by means of heat conduction, and convection heat transfer and heat radiation heat transfer are performed between a surface of the battery and an environment;

the parameters of the active material of the lithium-ion battery obtained in steps S2-S4 are taken into the model to acquire a simulated result of the thermal runaway of the lithium-ion battery, and the simulated result is compared with an experimental result of actual thermal runaway of the lithium-ion battery in step S1 to verify the feasibility of the model, specifically comprising the following steps: the lithium-ion battery is placed into an adiabatic accelerating rate calorimeter for the thermal runaway experiment, with a temperature sensitivity set as 0.02° C./min; and a temperature change of the lithium-ion battery during the thermal runaway process is measured by using a thermocouple, and a measured result is compared with a model result; a control equation and boundary conditions of the three-dimensional model for thermal runaway of the lithium-ion battery are as shown in Table 1;

TABLE 1
Control equation and boundary conditions of the model

| Physical | | |
|---|---|---|
| quantity | Control equation and boundary conditions | |
| Energy conservation equation | $ρ {C}_{p} \frac{\partial T}{\partial t} = λ {∇}^{2} T + {Q}_{total}$ | (1) |
| | Qₜₒₜₐₗ = Q_{caan} + Qₛₑₚ + Qₐₙₑₗₑ + Q_{ca} | (2) |
| | Qₓ = ΔHₓ · Kₓ · Wₓ | (3) |
| | $\frac{{dc}_{x}}{dt} = - {K}_{x} , {c}_{x , 0} = 1$ | (4) |
| | ${K}_{x} = {A}_{x} \cdot exp ( - \frac{{E}_{a , x}}{RT} ) \cdot f ( {c}_{x} )$ | (5) |
| | f(cₓ) = d · [(1 − cₓ)ª + p] · (cₓ^{b}) | (6) |
| Thermal boundary conditions | $- λ \frac{\partial T}{\partial n} = h ( T - {T}_{amb} )$ | (7) |
| | $- λ \frac{\partial T}{\partial n} = ϵ σ \cdot ( {T}^{4} - {T}_{amb}^{4} )$ | (8) |

S6: the battery is charged to different states of charge (SOC), namely 100% SOC, 80% SOC, 60% SOC, 40% SOC and 20% SOC respectively, by the charge-discharge meter. Steps S1-S5 are repeated under different SOC, and the influence of different SOC on the thermal runaway of the lithium-ion battery is studied.

### Example 1

Taking a commercial 2.6 Ah 18650 type NCM523/graphite lithium-ion battery as an example, a thermal runaway model for the battery is established and verified with an experimental result, and the present invention is fully described in detail. The method is not limited to this battery, but is also applicable to thermal runaway modeling for other batteries.

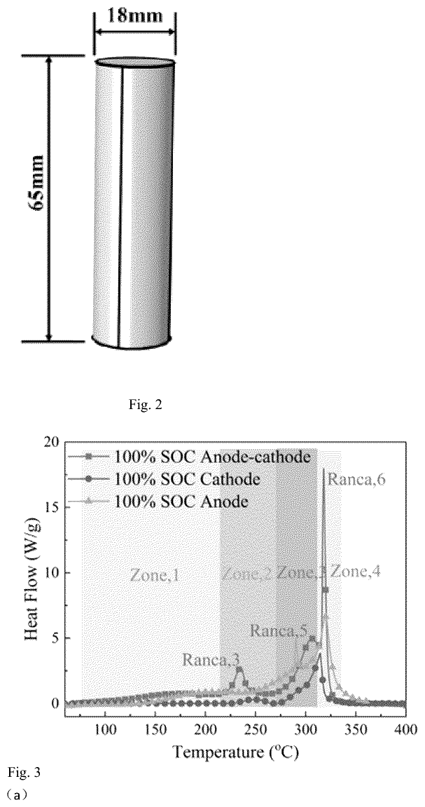

Simulated sizes of the battery in this example are shown in FIG. 2, with a length of the battery of 65 mm and a diameter of 18 mm. The establishment of the model is mainly divided into three parts: a DSC experiment, simulation and ARC experimental verification and analysis.

I. For the DSC Experimental Part:

(1) In this example, the batteries are firstly cycled three times by using Xinwei to determine parameters such as the capacity of the batteries, and the batteries with better performance are selected for later use; (2) the batteries are charged to 20% SOC, 40% SOC, 60% SOC, 80% SOC and 100% SOC respectively; (3) the batteries are placed into a glove box for disassembly, cathode terminals of the batteries are firstly removed by using a pipe wrench, in this process, it is necessary to pay attention that cathode tabs of the batteries are not in contact with shells of the batteries so as to avoid thermal runaway caused by a short circuit; then cathode active materials and anode active materials of the batteries under different SOC are scraped off by using a scraper, and put into a sample bag for later use; separators of the batteries are made to be powdery by using a pair of scissors or other grinding tools, and put into a sample bag for later use; the cathode active materials are mixed with the anode active materials in an equal proportion according to positive and anode active materials in a total battery, and denoted as A; the anode active materials are mixed with electrolytes in the equal proportion, and denoted as B; the cathode active materials are denoted as C; the separators are denoted as D; the electrolytes are denoted as E; cathodes are mixed with the electrolytes in the equal proportion, and denoted as F; and anodes are denoted as G; (4) experiments are performed on A, B, C, D, E, F and G under different SOC conditions by using a Mettler DSC under 20° C. min⁻¹ conditions respectively, and experimental results are shown in FIG. 3 and FIG. 5(d), it can be seen from the figures, the cathodes and the anodes can react with each other and generate a large amount of heat, the anodes and the electrolytes can also react with each other and generate a large amount of heat, while the cathodes and the electrolytes hardly react with each other or react with each other and generate a small amount of heat; therefore, A, B, C and D are the main heat sources in the thermal runaway process of the batteries in this model; (5) the DSC experiments are performed on A, B, C and D under different SOC at 10° C. min⁻¹, 15° C. min⁻¹, 20° C. min⁻¹ and 25° C. min⁻¹ temperature rise rates;

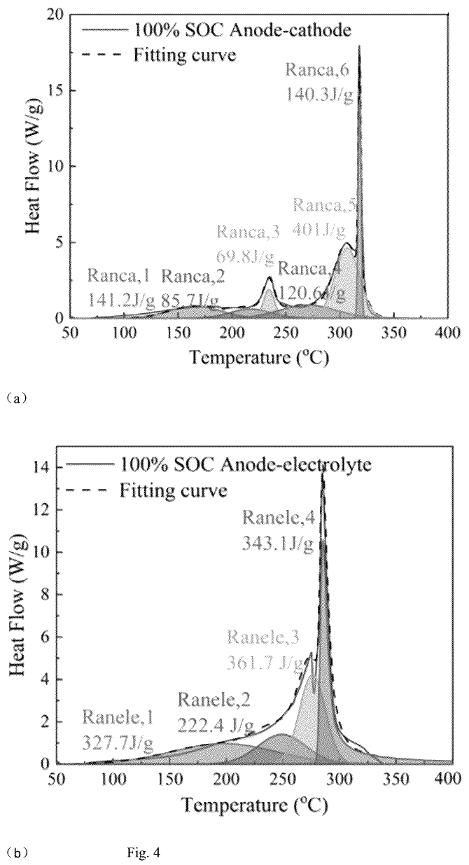

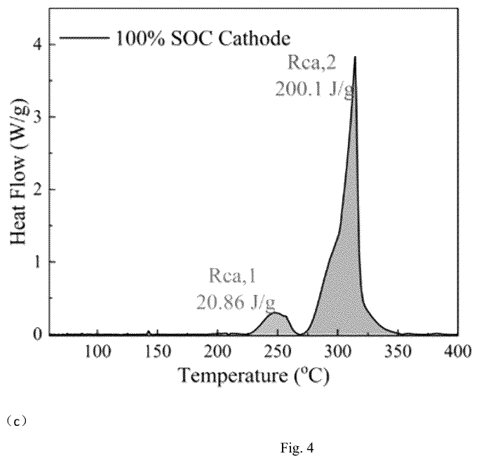

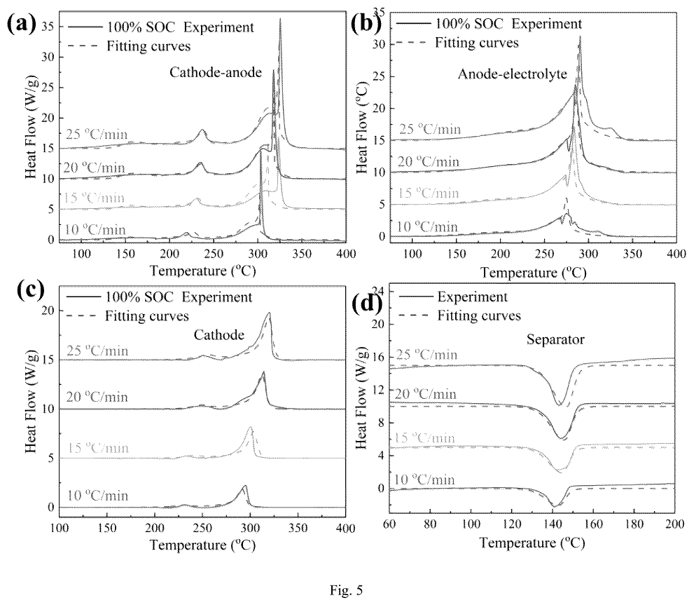

(6) peak differentiating and fitting are performed on heat flow curves of the four materials of the batteries A, B, C and D respectively by using a non-linear fitting method to obtain peak temperatures and reaction enthalpies of different peaks, and peak differentiating and fitting for the cathodes+the anodes, the anodes+the electrolytes, and the cathodes under 100% SOC at 20° C. min⁻¹ temperature rise rate are shown in FIG. 4;

(7) DSC experimental data is processed by using a Kissinger equation to obtain pre-exponential factors and activation energy of the materials of the batteries; and

(8) The heat flow curves of the batteries are fit by using a genetic algorithm to obtain reaction orders a, b and constants p, d of the batteries; and fitting results and experimental results of the cathodes+the anodes, the anodes+the electrolytes, the cathodes, and the separators under 100% SOC conditions are shown in FIG. 4.

II. The Simulation Part:

(1) Based on the parameters acquired by the DSC experiments, a thermal runaway reaction equation of the battery is established, and then a three-dimensional thermal runaway reaction model for the battery is further established based on COMSOL software; and (2) a three-dimensional heat transfer model for the battery is established based on the COMSOL software. Tables 3-18 show model parameters for 100% SOC, 80% SOC, 60% SOC, 40% SOC and 20% SOC in this example.

The parameters and meanings presented herein are shown in Table 2.

TABLE 2
Meanings of parameters

| Symbols | Interpretation |
|---|---|
| t | Time, s; |
| R | Ideal gas state constant, 8.314, J/mol/K; |
| T | Temperature of the material of the battery, K; |
| Cₚ | Specific heat capacity, J/kg/K; |
| T_{amb} | Ambient temperature, K; |
| Qₜₒₜₐₗ | Total generated heat, W/m³; |
| Q_{caan} | Heat generated by the mixed material of the |
| | cathode and the anode, W/m³; |
| λ | Heat conductivity of the material |
| | of the battery, W/m/K; |
| Qₛₑₚ | Heat generated by the separator of the battery, |
| | W/m³; |
| Qₐₙₑₗₑ | Heat generated by the mixed material of the |
| | anode and the electrolyte of the battery, W/m³; |
| Q_{ca} | Heat generated by the cathode material of the |
| | battery, W/m³; |
| Kₓ | Decomposition reaction rate of the material of |
| | the battery, 1/s; |
| wₓ | Mass fraction of the material of the battery, |
| | kg/m³; |
| ΔHₓ | Reaction enthalpy of the material |
| | of the battery, J/g; |
| Cx | Reaction concentration of the material of the |
| | battery; |
| b | Reaction order; |
| p | Reaction order; |
| d | Reaction order; |
| h | Heat transfer coefficient, W/m²/K; |
| ∈ | Emissivity coefficient; |
| σ | Stefan-Boltzmann constant |
| | 5.67 × 10⁻⁸ W m⁻² K⁻⁴; |
| Aₓ | pre-exponential factor of the material of the |
| | battery; |
| C_{x,0} | Initial value of reaction concentration of |
| | material of battery |
| Caan1 | Cathode + anode reaction peak 1 |
| Caan2 | Cathode + anode reaction peak 2 |
| Caan3 | Cathode + anode reaction peak 3 |
| Caan4 | Cathode + anode reaction peak 4 |
| Caan5 | Cathode + anode reaction peak 5 |
| Caan6 | Cathode + anode reaction peak 6 |
| Anele1 | Electrolyte + anode reaction peak 1 |
| Anele2 | Electrolyte + anode reaction peak 2 |
| Anele3 | Electrolyte + anode reaction peak 3 |
| Anele4 | Electrolyte + anode reaction peak 4 |
| Sep | Separator reaction peak |
| Ca1 | Cathode reaction peak 1 |
| Ca2 | Cathode reaction peak 2 |

TABLE 3
Parameters of 100% SOC battery model 1

| Parameters | Caan1 | Caan2 | Caan3 | Caan4 | Caan5 | Caan6 |
|---|---|---|---|---|---|---|
| Pre-exponential | 3.98634E+26 | 1.10855E+19 | 3.02926E+14 | 1.29013E+13 | 821050062 | 93159283.34 |
| factor | | | | | | |
| [1/s] | | | | | | |
| Reaction | 141.15 | 85.66 | 69.8 | 120.6 | 401 | 140.27 |
| enthalpy [J/g] | | | | | | |
| Activation | 2.5E+05 | 1.93E+05 | 1.66E+05 | 1.53E+05 | 1.20E+05 | 1.12E+05 |
| energy | | | | | | |
| [J/mol] | | | | | | |
| Reaction | 6.1576 | 2.6349 | 0.9726 | 2.2139 | 1.2643 | 0.1932 |
| order a | | | | | | |
| Reaction | 3.4184 | 5.7262 | 0.9149 | 0 | 0.6755 | 2.5567 |
| order b | | | | | | |
| Reaction | 1.4820 | 0.5904 | 0.0303 | 0 | 0.0004 | 0.0247 |
| order p | | | | | | |
| Reaction | 169.9320 | 2.2465 | 34.1718 | 1 | 3.6017 | 14.1026 |
| order d | | | | | | |
| Active | 3.03E+02 | 3.03E+02 | 7.26E+02 | 1.51E+03 | 1.51E+03 | 1.51E+03 |
| substance | | | | | | |
| [kg/m³] | | | | | | |

TABLE 4
Parameters of 100% SOC battery model 2

| Param- | | | | |
|---|---|---|---|---|
| eters | Anele1 | Anele2 | Anele3 | Anele4 |
| Pre- | 2717550891 | 147823841.2 | 28560873.82 | 7.24508E+11 |
| expo- | | | | |
| nential | | | | |
| factor | | | | |
| [1/s] | | | | |
| Reaction | 327.68 | 222.39 | 361.71 | 295.91 |
| enthalpy | | | | |
| [J/g] | | | | |
| Activation | 98692.84641 | 94784.93606 | 9.05E+04 | 1.46E+05 |
| energy | | | | |
| [J/mol] | | | | |
| Reaction | 3.8633 | 1.7547 | 1.2552 | 1.1543 |
| order a | | | | |
| Reaction | 0 | 1.3738 | 0.8535 | 1.6388 |
| order b | | | | |
| Reaction | 0 | 0.2293 | 0.0082 | 0.0209 |
| order p | | | | |
| Reaction | 1 | 0.7281 | 0.7169 | 10.3001 |
| order d | | | | |
| Active | 7.26E+02 | 7.26E+02 | 7.26E+02 | 7.26E+02 |
| substance | | | | |
| [kg/m³] | | | | |

TABLE 5
Parameters of 100% SOC battery model 3

| Parameters | Sep | Ca1 | Ca2 |
|---|---|---|---|
| Pre-exponential factor | 2.0048E+44 | 5.55596E+13 | 795683294.5 |
| [1/s] | | | |
| Reaction enthalpy | −159.51 | 20.86 | 200.1 |
| [J/g] | | | |
| Activation energy | 3.48E+05 | 1.54E+05 | 1.21E+05 |
| [J/mol] | | | |
| Reaction order a | 1.7218 | 0.6247 | 0.1364 |
| Reaction order b | 4.2202 | 0 | 0.9546 |
| Reaction order p | 0.4471 | 0 | 0.6571 |
| Reaction order d | 0.0321 | 1 | 0.3695 |
| Active substance | 1.63E+02 | 6.05E+02 | 6.05E+02 |
| [kg/m³] | | | |

TABLE 6
Parameters of 80% SOC battery model 1

| Parameters | Caan1 | Caan2 | Caan3 | Caan4 | Caan5 |
|---|---|---|---|---|---|
| Pre-exponential | 2.45733E+28 | 1.45907E+16 | 1007495658 | 5.25869E+20 | 1.92909E+24 |
| factor [1/s] | | | | | |
| Reaction | 82 | 196.15 | 113.2 | 363.7 | 40.71 |
| enthalpy [J/g] | | | | | |
| Activation | 2.58E+05 | 1.51E+05 | 1.068E+05 | 2.42E+05 | 2.90E+05 |
| energy [J/mol] | | | | | |
| Reaction order | 8.1321 | 7.2838 | 1.2056 | 1.6603 | 1.1294 |
| a | | | | | |
| Reaction order | 3.1472 | 7.5607 | 1.0501 | 0.1175 | 1.3205 |
| b | | | | | |
| Reaction order | 27.1997 | 0.0063 | 0.0037 | 0 | 0.0101 |
| p | | | | | |
| Reaction order | 27.1433 | 10.2929 | 3.5526 | 1.1358 | 10.7350 |
| d | | | | | |
| Active | 3.03E+02 | 7.26E+02 | 1.51E+03 | 1.51E+03 | 1.51E+03 |
| substance | | | | | |
| [kg/m³] | | | | | |

TABLE 7
Parameters of 80% SOC battery model 2

| Parameters | Anele1 | Anele2 | Anele3 | Anele4 |
|---|---|---|---|---|
| Pre- | 5.48776E+11 | 117056492.6 | 235178067.6 | 39791171592 |
| exponential | | | | |
| factor [1/s] | | | | |
| Reaction | 415.3 | 390.6 | 165.9 | 129.76 |
| enthalpy | | | | |
| [J/g] | | | | |
| Activation | 1.1E+05 | 1.15E+05 | 1E+05 | 1.33E+05 |
| energy | | | | |
| [J/mol] | | | | |
| Reaction | 5.5790 | 2.2918 | 1.1960 | 0.0888 |
| order a | | | | |
| Reaction | 6.6814 | 0.4887 | 1.1712 | 2.8098 |
| order b | | | | |
| Reaction | 0.0182 | 0.0018 | 0.0036 | 0.1763 |
| order p | | | | |
| Reaction | 2.4133 | 37.2361 | 1.6069 | 3.5844 |
| order d | | | | |
| Active | 7.26E+02 | 7.26E+02 | 7.26E+02 | 7.26E+02 |
| substance | | | | |
| [kg/m³] | | | | |

TABLE 8
Parameters of 80% SOC battery model 3

| Parameters | Sep | Ca1 |
|---|---|---|
| Pre-exponential factor [1/s] | 2.0048E+44 | 1.158E+12 |
| Reaction enthalpy [J/g] | −159.51 | 195 |
| Activation energy [J/mol] | 3.48E+05 | 148081.6517 |
| Reaction order a | 1.7218 | 1.2343 |
| Reaction order b | 4.2202 | 1.4068 |
| Reaction order p | 0.4471 | 0.0109 |
| Reaction order d | 0.0321 | 0.9154 |
| Active substance [kg/m³] | 1.63E+02 | 6.05E+02 |

TABLE 9
Parameters of 60% SOC battery model 1

| Parameters | Caan1 | Caan2 | Caan3 | Caan4 | Caan5 |
|---|---|---|---|---|---|
| Pre-exponential | 8.68893E+22 | 3.36649E+14 | 1347317091 | 2.05353E+13 | 4.08628E+21 |
| factor [1/s] | | | | | |
| Reaction | 134.95 | 97.4 | 120.8 | 328.8 | 32.1 |
| enthalpy [J/g] | | | | | |
| Activation | 2.05E+05 | 1.4E+05 | 1.23E+05 | 1.63E+05 | 2.56E+05 |
| energy [J/mol] | | | | | |
| Reaction order | 7.7895 | 3.9008 | 1.0221 | 1.2938 | 0.9718 |
| a | | | | | |
| Reaction order | 6.1035 | 6.0931 | 0.9753 | 0.5795 | 1.5671 |
| b | | | | | |
| Reaction order | 0.0252 | 0.0822 | 0.0076 | 0 | 0.0133 |
| p | | | | | |
| Reaction order | 150.1046 | 0.1587 | 42.8708 | 2.4516 | 10.2198 |
| d | | | | | |
| Active | 3.03E+02 | 7.26E+02 | 1.51E+03 | 1.51E+03 | 1.51E+03 |
| substance | | | | | |
| [kg/m³] | | | | | |

TABLE 10
Parameters of 60% SOC battery model 2

| Parameters | Anele1 | Anele2 | Anele3 | Anele4 |
|---|---|---|---|---|
| Pre-exponential | 2.8925E+11 | 896572503.2 | 657466984.8 | 1.02692E+12 |
| factor [1/s] | | | | |
| Reaction | 328.5 | 303.5 | 313.6 | 118.4 |
| enthalpy [J/g] | | | | |
| Activation | 115600.564 | 109418.234 | 111507.368 | 146609.076 |
| energy [J/mol] | | | | |
| Reaction order a | 4.5229 | 1.33115 | 0.8958 | 1.7897 |
| Reaction order b | 6.3980 | 0.4932268 | 0.6293 | 1.5679 |
| Reaction order p | 0.0481 | 0.0000120828 | 0 | 0.0048 |
| Reaction order d | 9.8520 | 1.5899 | 1.7982 | 15.7588 |
| Active substance | 7.26E+02 | 7.26E+02 | 7.26E+02 | 7.26E+02 |
| [kg/m³] | | | | |

TABLE 11
Parameters of 60% SOC battery model 3

| Parameters | Sep | Ca1 |
|---|---|---|
| Pre-exponential factor [1/s] | 2.0048E+44 | 422708642.2 |
| Reaction enthalpy [J/g] | −159.51 | 190.86 |
| Activation energy [J/mol] | 3.48E+05 | 1.18E+05 |
| Reaction order a | 1.7218 | 0.5928 |
| Reaction order b | 4.2202 | 2.3156 |
| Reaction order p | 0.4471 | 0.0255 |
| Reaction order d | 0.0321 | 11.8629 |
| Active substance [kg/m³] | 1.63E+02 | 6.05E+02 |

TABLE 12
Parameters of 40% SOC battery model 1

| Parameters | Caan1 | Caan2 | Caan3 | Caan4 |
|---|---|---|---|---|
| Pre- | 9.07847E+26 | 1.46506E+13 | 1291892633 | 7.10689E+11 |
| exponential | | | | |
| factor [1/s] | | | | |
| Reaction | 280 | 149.8 | 102.2 | 207 |
| enthalpy | | | | |
| [J/g] | | | | |
| Activation | 2.7E+05 | 1.25E+05 | 1.1E+05 | 1.48E+05 |
| energy | | | | |
| [J/mol] | | | | |
| Reaction | 16.0329 | 3.1954 | 1.0922 | 2.5800 |
| order a | | | | |
| Reaction | 182.0667 | 6.1839 | 1.2294 | 1.3511 |
| order b | | | | |
| Reaction | 454.6798 | 0.0763 | 0.0124 | 0.0104 |
| order p | | | | |
| Reaction | 454.3 898 | 0.0230 | 1.7790 | 8.7991 |
| order d | | | | |
| Active | 3.03E+02 | 7.26E+02 | 1.51E+03 | 1.51E+03 |
| substance | | | | |
| [kg/m³] | | | | |

TABLE 13
Parameters of 40% SOC battery model 2

| Parameters | Anele1 | Anele2 | Anele3 | Anele4 |
|---|---|---|---|---|
| Pre- | 132338304.2 | 1066231042 | 1.07891E+11 | 949135811.5 |
| exponential | | | | |
| factor [1/s] | | | | |
| Reaction | 257.45 | 162.93 | 110.6 | 40.25 |
| enthalpy | | | | |
| [J/g] | | | | |
| Activation | 99556.27607 | 109089.812 | 134736.839 | 116394.818 |
| energy | | | | |
| [J/mol] | | | | |
| Reaction | 2.4856 | 1.2642 | 1.1735 | 0.1328 |
| order a | | | | |
| Reaction | 5.6350 | 0.7972 | 1.2043 | 4.085 |
| order b | | | | |
| Reaction | 0.3258 | 0.0059 | 0.0046 | 0.2311 |
| order p | | | | |
| Reaction | 13.0083 | 2.0219 | 8.4594 | 3.1093 |
| order d | | | | |
| Active | 7.26E+02 | 7.26E+02 | 7.26E+02 | 7.26E+02 |
| substance | | | | |
| [kg/m³] | | | | |

TABLE 14
Parameters of 40% SOC battery model 3

| Parameters | Sep | Ca1 |
|---|---|---|
| Pre-exponential factor [1/s] | 2.0048E+44 | 1.43426E+19 |
| Reaction enthalpy [J/g] | −159.51 | 0.0453 |
| Activation energy [J/mol] | 3.48E+05 | 2.30E+05 |
| Reaction order a | 1.7218 | 0.6338 |
| Reaction order b | 4.2202 | 1.2284 |
| Reaction order p | 0.4471 | 0.0453 |
| Reaction order d | 0.0321 | 4.5225 |
| Active substance [kg/m³] | 1.63E+02 | 6.05E+02 |

TABLE 15
Parameters of 20% SOC battery model 1

| Param- | Caan1 | Caan2 | Caan3 | Caan4 |
|---|---|---|---|---|
| eters | | | | |
| Pre- | 7.10075E+16 | 86594392480 | 7.65574E+11 | 3.89456E+12 |
| expo- | | | | |
| nential | | | | |
| factor | | | | |
| [1/s] | | | | |
| Reaction | 79.28 | 111.9 | 116.5 | 34.7 |
| enthalpy | | | | |
| [J/g] | | | | |
| Activa- | 1.8E+05 | 1.29E+05 | 1.45E+05 | 1.59E+05 |
| tion | | | | |
| energy | | | | |
| [J/mol] | | | | |
| Reaction | 6.1059 | 3.3250 | 1.2405 | 1.2673 |
| order a | | | | |
| Reaction | 3.0613 | 5.6059 | 0.7415 | 0.7283 |
| order b | | | | |
| Reaction | 38.6689 | 0.1941 | 0.0019 | 0.0028 |
| order p | | | | |
| Reaction | 87.6900 | 15.9584 | 4.0627 | 4.2341 |
| order d | | | | |
| Active | 3.03E+02 | 7.26E+02 | 1.51E+03 | 1.51E+03 |
| sub- | | | | |
| stance | | | | |
| [kg/m³] | | | | |

TABLE 16
Parameters of 20% SOC battery model 2

| Parameters | Anele1 | Anele2 | Anele3 |
|---|---|---|---|
| Pre-exponential | 11149111061 | 7830395794 | 4.80336E+11 |
| factor | | | |
| [1/s] | | | |
| Reaction enthalpy | 302.7 | 153 | 92.2 |
| [J/g] | | | |
| Activation energy | 99378.1526 | 128092.056 | 140321.7465 |
| [J/mol] | | | |
| Reaction order a | 3.7994 | 1.1057 | 1.1789 |
| Reaction order b | 3.9330 | 0.4936 | 0.8556 |
| Reaction order p | 0.0959 | 0.0707 | 0.0010 |
| Reaction order d | 0.6135 | 17.2621 | 5.6549 |
| Active substance | 7.26E+02 | 7.26E+02 | 7.26E+02 |
| [kg/m³] | | | |

TABLE 17
Parameters of 20% SOC battery model 3

| Parameters | Sep | Ca1 |
|---|---|---|
| Pre-exponential factor [1/s] | 2.0048E+44 | 1993236.973 |
| Reaction enthalpy [J/g] | −159.51 | 167.4 |
| Activation energy [J/mol] | 3.48E+05 | 9.23E+04 |
| Reaction order a | 1.7218 | 0.0943 |
| Reaction order b | 4.2202 | 2.8386 |
| Reaction order p | 0.4471 | 0.2174 |
| Reaction order d | 0.0321 | 3.1016 |
| Active substance [kg/m³] | 1.63E+02 | 6.05E+02 |

TABLE 18
Thermophysical parameters of the battery

| | | Specific | | | | Heat | |
|---|---|---|---|---|---|---|---|
| | | heat | Heat | Heat | Heat | transfer | |
| | Density | capacity | conductivity | conductivity | conductivity | coefficient | |
| Parameters | [kg/m³] | [J/kg/K] | x[W/m/K] | y[W/m/K] | z[W/m/K] | [w/m²/K] | Emissivity |
| Battery | 2637.63 | 1099.8 | 1.369 | 1.369 | 37.12 | 0 | 0 |

III. The ARC Experimental Validation and Analysis Part:

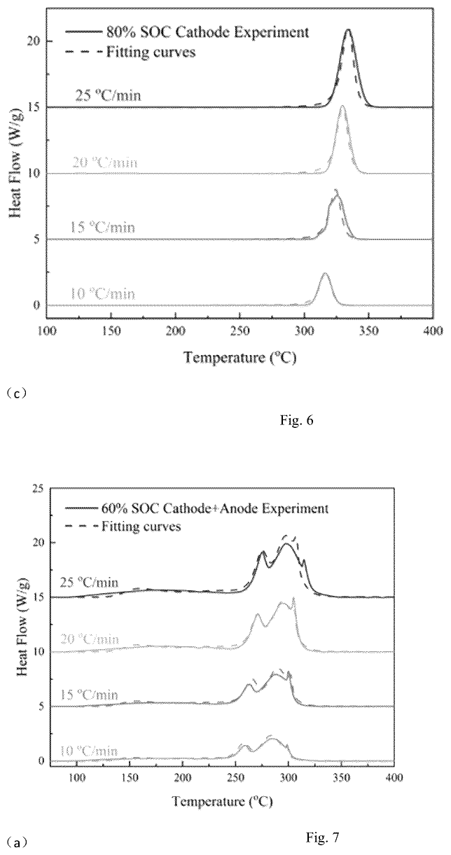

(1) The batteries with good performance are selected and charged to 20% SOC, 40% SOC, 60% SOC, 80% SOC and 100% SOC by using a Xinwei charge and discharge apparatus to prepare for later use; (2) the batteries are placed into a THT ES-ARC experimental cavity, and K-type battery thermocouples are fixed on surfaces of the batteries by using high-temperature resistant tapes; (3) H-W-S program setting for an ARC apparatus is performed, namely, a heating-waiting-searching process, a searching value is set as 0.02° C./min, namely, when a self-heating temperature rise rate of the battery reaches 0.02° C./min, the ARC program enters an adiabatic mode, and an initial experimental temperature is set as 50° C.; (4) the end of the ARC experiment is waited; (5) an ARC experimental result is compared with a simulation calculated result to verify the effectiveness and accuracy of the model, and comparison of experimental and model results under 20% SOC, 40% SOC, 60% SOC, 80% SOC and 100% SOC are shown in FIG. 5; and (6) four types of heat generation curves under different SOC conditions can be obtained by the model, the proportion of the four types of heat generation in the thermal runaway process can be obtained, and the results are shown in FIG. 6. Tables 3-18 show the model parameters for 100% SOC, 80% SOC, 60% SOC, 40% SOC and 20% SOC in this example, and these parameters are mainly obtained through steps S1-S4.

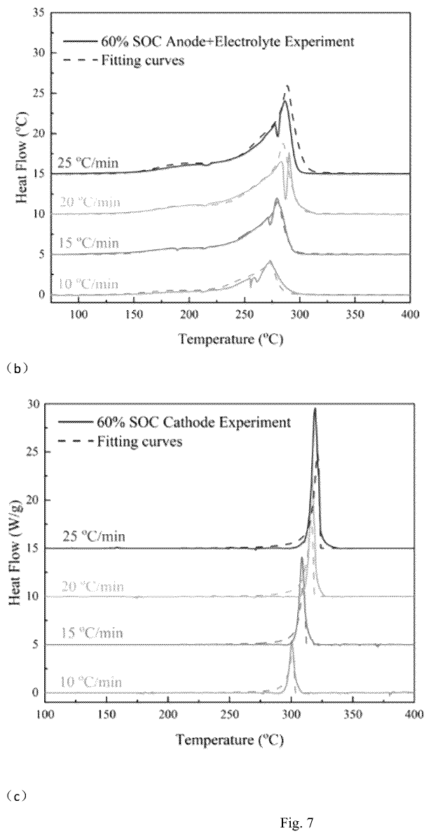

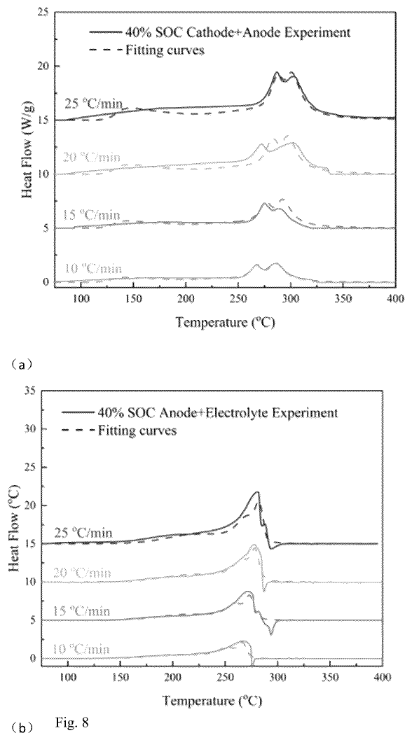

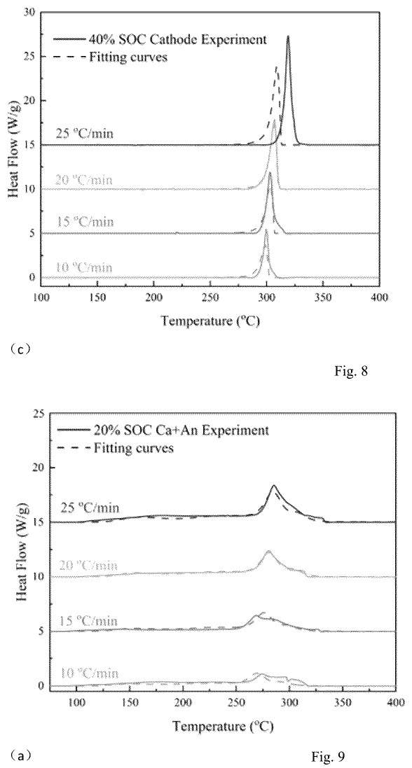

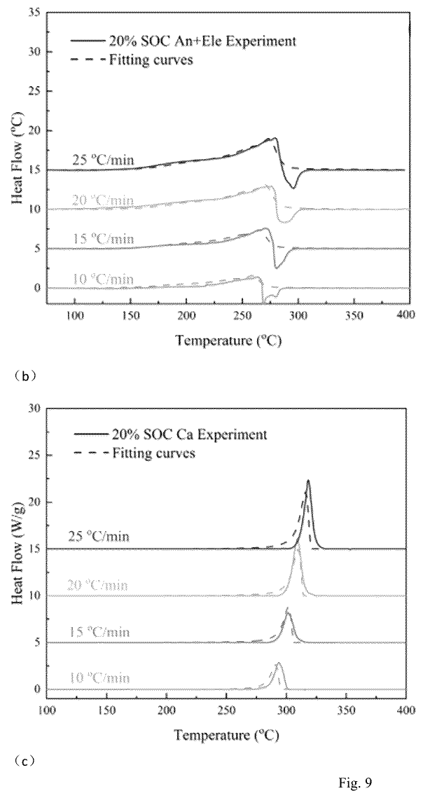

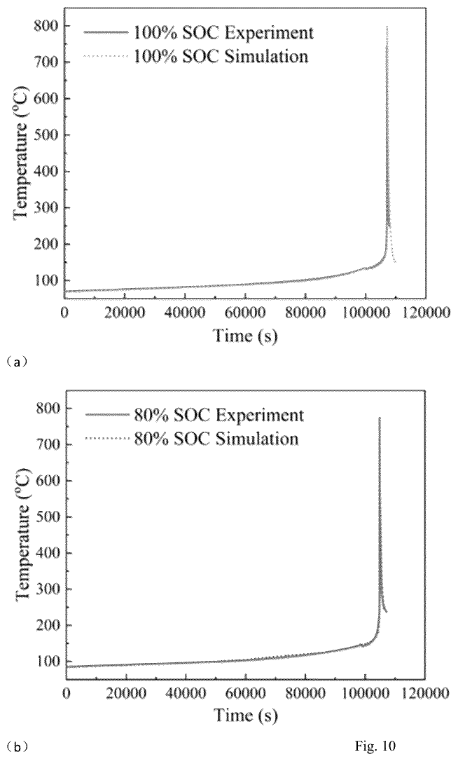

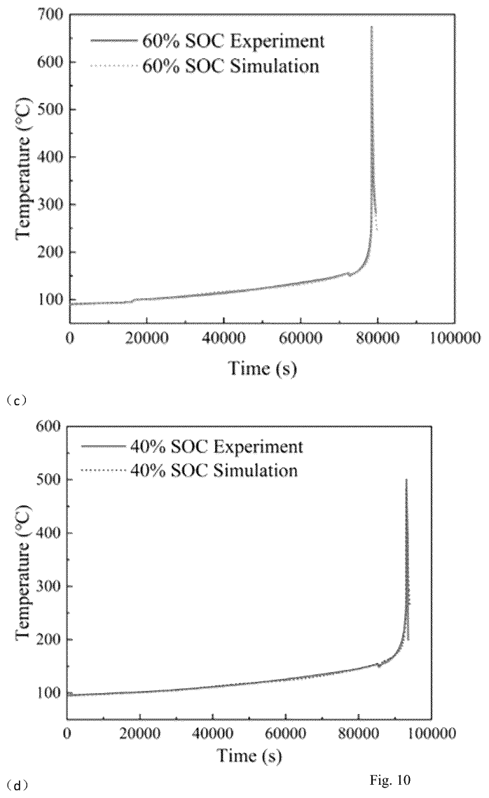

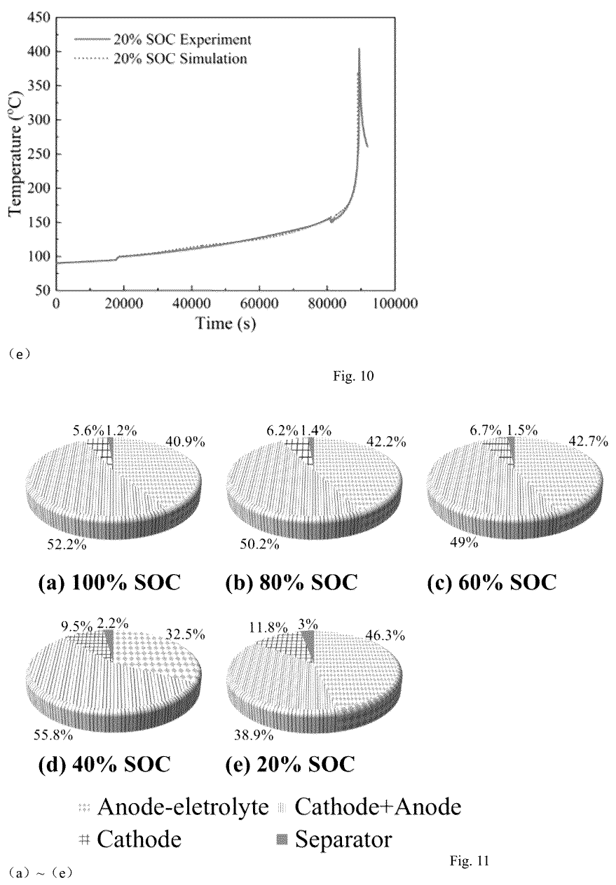

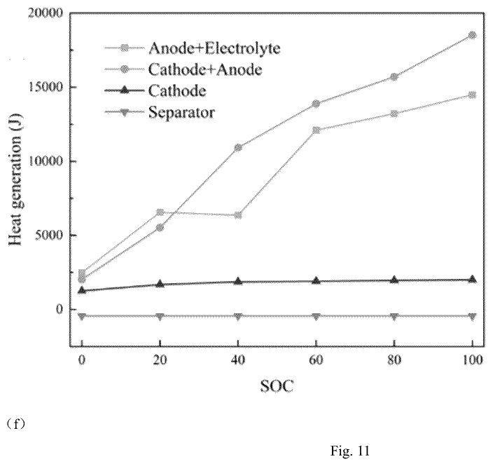

FIG. 3(a) shows the heat flow curves of the cathode+the anode, the cathode, and the anode of the battery. It can be seen from the figure that there are three obvious exothermic peaks in the cathode+the anode of the battery, and by comparing the heat flow curves of the anode and the cathode, it can be seen that a violent chemical reaction can occur between the materials of the cathode and the anode, and heat is released. FIG. 3(b) shows the heat flow curves of the anode+the electrolyte, the anode, and the electrolyte of the battery, and it can be seen from the figure that the chemical reaction can also occur between the anode and the electrolyte and heat is released. FIG. 3(c) shows the heat flow curves of the cathode+the electrolyte, the cathode, and the electrolyte, and it can be seen from the figure that there are mainly three reaction peaks for the cathode+the electrolyte, the first reaction peak is mainly caused by the decomposition of a solid permeable interface film of the cathode material, which is similar to the decomposition of a solid electrolyte phase interface film on a surface of the anode; the second reaction peak is caused by heat adsorption of the electrolyte, which can be obtained from the reaction peak of the electrolyte; and the third reaction peak is caused by the decomposition of the cathode material, which can be seen from the reaction peak of the cathode. However, by comparing the reaction peaks of the cathode+the electrolyte, the electrolyte, and the cathode, it can be seen that the reaction between the cathode and the electrolyte of the battery is less, so that the reaction between the cathode and the electrolyte is ignored in the modeling process. FIG. 4(a) and FIG. 4(b) show the peak division and fitting curves of the cathode+the anode, and the anode+the electrolyte of the battery under 100% SOC, while the peak type of the cathode is divided, so that the peak division is not needed, and FIG. 4(c) shows the peak reaction enthalpy of the cathode. FIGS. 5-9 show comparison results of the heat flow curves of the cathode+the anode, the anode+the electrolyte, the cathode, and the separator under 100% SOC, 80% SOC, 60% SOC, 40% SOC and 20% SOC respectively, and the fitting curves obtained by using the genetic algorithm. It can be seen from the figures that the fitting degree of the experimental results and the fitting results is good, and a, b, p and d values of the battery under different SOC can be obtained by this method. FIG. 10 shows the experimental results and simulated results of ARC thermal runaway of the battery under different SOC, and it can be seen from the figure that the matching degree of the experimental results and the simulated results is good, and can well reflect the whole process of the battery from self-heating to thermal runaway and then cooling. FIG. 11 shows the proportion of four types of main heat generation in the thermal runaway process of the battery under different SOC calculated by the model and the specific quantity of heat generation, and the main quantity of heat generation of each heat source can be understood from the model. It can be seen that the cathode+the anode, and the anode+the electrolyte are the main heat sources in the thermal runaway process of the battery under different SOC, but the heat generation proportion of the cathode and the separator can increase with the decrease of SOC.

From the above analysis, it can be concluded that as the SOC decreases, the risk of thermal runaway of the battery gradually decreases. The main heat sources for thermal runaway of the battery are the reaction between the cathode+the anode, and the anode+the electrolyte. However, with the decrease of SOC, the heat generation proportion of the cathode and the separator also increases. Through this modeling method, the reaction process of the battery in the thermal runaway process can be actually reflected to obtain the heat generation proportion in different reactions.

The examples described above are merely representative of the embodiments of the present application and are not to be construed as limiting the scope of the invention patent. It should be noted that a person skilled in the art could make several changes and modifications without departing from the concept of the present application, and these changes and modifications fall within the protection scope of the present application.

1. A three-dimensional modeling method for thermal runaway of a lithium-ion battery under different state of charge conditions based on a differential scanning calorimeter experiment, characterized by comprising the following steps:
S1: acquiring an active material of the lithium-ion battery with a set charge value, and performing the differential scanning calorimeter experiment on the active material to acquire heat flow curves of the active material of the lithium-ion battery at different temperature rise rates respectively;
S11: charging the lithium-ion battery to a set electric quantity value by utilizing a charge and discharge meter, and then placing the lithium-ion battery into a glove box for disassembly to acquire a cathode active material, a anode active material, an electrolyte and a separator of the battery, and making the cathode active material, the anode active material and the separator into powder; and
mixing the cathode active material with the anode active material in an equal proportion according to positive and anode active materials in a total battery, and denoting as A; and mixing the anode active material with the electrolyte in the equal proportion, and denoting as B; denoting the cathode active material as C; denoting the separator as D; denoting the electrolyte as E; mixing a cathode with the electrolyte in the equal proportion, and denoting as F; and denoting a anode as G; and
S12: placing the four substances A, B, C and D in step S11 into a DSC apparatus by utilizing a standard aluminium crucible respectively, and performing experiments at four temperature rise rates of 10° C. min⁻¹, 15° C. min⁻¹, 20° C. min⁻¹ and 25° C. min⁻¹ respectively;
S2: dividing the heat flow curve of the battery into a plurality of reaction peaks by using a non-linear fitting method to obtain a reaction enthalpy of each peak of the battery;
S3: analyzing the heat flow curves acquired in step S1 by utilizing a Kissinger equation to obtain activation energy and a pre-exponential factor of the active material of the lithium-ion battery;
S4: fitting the heat flow curves of the material of the battery by using a genetic algorithm to obtain a reaction order of the active material of the lithium-ion battery;
in order to fit the heat flow curves of the material of the battery by using the genetic algorithm to obtain the reaction order of the material of the battery, a heat generation formula of the lithium-ion battery used is as follows:
$${Qm}_{x} = Δ {H}_{x} \cdot {K}_{x} \cdot m ;$$
$${K}_{x} = {A}_{x} \cdot exp ( - \frac{{E}_{a , x}}{RT} ) \cdot f ( {c}_{x} ) ;$$
$$\frac{{dc}_{x}}{dt} = - {K}_{x} , {c}_{x , 0} = 1 ;$$
$$f ( {c}_{x} ) = d \cdot [ {( 1 - {c}_{x} )}^{a} + p ] \cdot ( {c}_{x}^{b} ) ;$$
in the above formula, Kₓ is a decomposition reaction rate of the material of the battery, 1/s; cₓ is a reaction concentration of the material of the battery; ΔHₓ is the reaction enthalpy of the material of the battery, J/g; Qmₓ is heat generated by the material of the battery, W; c_{x,0} is an initial value of the reaction concentration of the material of the battery; a, b are reaction orders; p, d are reaction orders; T is a temperature of the material of the battery, K; and m is a mass of the material of the battery, and is normalized to 1 mg in this formula;
a peak temperature and the reaction enthalpy of the battery can be obtained by step S2;
the pre-exponential factor and activation energy of the material of the battery can be obtained by step S3; an objective function is set as Qmₓ and variables are set as a, b, p, d, the above formula is put into an MATLAB program, and a Qmₓ value of the best fitting objective function is obtained by utilizing the genetic algorithm to obtain a, b, p, d values best matched with the Qmₓ value;
S5: establishing a thermal runaway model for the lithium-ion battery, and taking parameters of the active material of the lithium-ion battery obtained in steps S2-S4 into the model to acquire a simulated result of the thermal runaway of the lithium-ion battery, and comparing the simulated result with an experimental result of actual thermal runaway of the lithium-ion battery in step S1 to verify the feasibility of the model;
a control equation and boundary conditions of the three-dimensional model for thermal runaway of the lithium-ion battery are as follows;
an energy conservation equation:
$$ρ {C}_{p} \frac{\partial T}{\partial t} = λ {∇}^{2} T + {Q}_{total} ;$$
$${Q}_{total} = {Q}_{caan} + {Q}_{sep} + {Q}_{anele} + {Q}_{ca} ;$$
$${Q}_{x} = Δ {H}_{x} \cdot {K}_{x} \cdot {W}_{x} ;$$
$$\frac{{dc}_{x}}{dt} = - {K}_{x} , {c}_{x , 0} = 1 ;$$
$${K}_{x} = {A}_{x} \cdot exp ( - \frac{{E}_{a , x}}{RT} ) \cdot f ( {c}_{x} ) ;$$
$$f ( {c}_{x} ) = d \cdot [ {( 1 - {c}_{x} )}^{a} + p ] \cdot ( {c}_{x}^{b} ) ;$$
the boundary conditions:
$$- λ \frac{\partial T}{\partial n} = h ( T - {T}_{amb} ) ;$$
$$- λ \frac{\partial T}{\partial n} = ϵσ \cdot ( {T}^{4} - {T}_{amb}^{4} ) ;$$
in the above formula, the meaning of each parameter is as follows:
t time, s;
R ideal gas state constant, 8.314, J/mol/K;
T temperature of the material of the battery, K;
Cₚ specific heat capacity, J/kg/K;
T_{amb} ambient temperature, K;
Qₜₒₜₐₗ total generated heat, W/m³;
Q_{caan} heat generated by the mixed material of the cathode and the anode, W/m³;
λ heat conductivity of the material of the battery, W/m/K;
Qₛₑₚ heat generated by the separator of the battery, W/m₃;
Qₐₙₑₗₑ heat generated by the mixed material of the anode and the electrolyte of the battery, W/m³;
Q_{ca} heat generated by the cathode material of the battery, W/m³;
Kₓ decomposition reaction rate of the material of the battery, 1/s;
Wₓ mass fraction of the material of the battery, kg/m₃;
ΔHₓ reaction enthalpy of the material of the battery, J/g;
Cₓ reaction concentration of the material of the battery;
b reaction order;
p reaction order;
d reaction order;
h heat transfer coefficient, W/m²/K;
∈ emissivity coefficient;
σ Stefan-Boltzmann constant 5.67×10⁻⁸ W m⁻² K⁻⁴;
Aₓ pre-exponential factor of the material of the battery;
C_{x,0} initial value of reaction concentration of the material of the battery; and
S6: changing a state of charge of the lithium-ion battery, respectively repeating steps S1-S5 under different states of charge, and studying the influence of different states of charge on the thermal runaway of the lithium-ion battery.

2. The three-dimensional modeling method for thermal runaway of a lithium-ion battery under different state of charge conditions based on a differential scanning calorimeter experiment according to claim 1, characterized in that in the step S3, activation energy and pre-exponential factors of different reaction peaks are respectively obtained by using Kissinger's equation fitting based on reaction peak temperatures at different temperature rise rates and reaction enthalpies of different peaks, wherein Kissinger's equation is as follows:
$$ln ( \frac{{α}_{i}}{{T}_{i}^{2}} ) = ln ( \frac{{A}_{x}}{{E}_{a , x}} ) - \frac{{E}_{a , x}}{{RT}_{i}} , i = 1 , 2 , 3 ... u ;$$
in the above formula, R is the ideal gas state constant, 8.314, J/mol/K; Aₓ is the pre-exponential factor of the material of the battery; E_{a,x} is the activation energy of the material of the battery; Ti is the peak temperature; u is a serial number of the changing temperature rise rate; and α is the temperature rise rate;
$$ln ( \frac{{α}_{i}}{{T}_{i}^{2}} )$$
is taken as a dependent variable,
$$\frac{1}{{T}_{i}}$$
is taken as an independent variable, linear fitting is performed, the obtained slope of a straight line is multiplied by R to obtain the activation energy E_{a,x} of the reaction peak, while the reaction pre-exponential factor of the reaction peak can be obtained by an intercept
$$ln ( \frac{{A}_{x}}{{E}_{a , x}} )$$
of the straight line.

3. The three-dimensional modeling method for thermal runaway of a lithium-ion battery under different state of charge conditions based on a differential scanning calorimeter experiment according to claim 1, characterized in that in the step S5, the three-dimensional model is established according to actual sizes of the lithium-ion battery, and the model comprises a three-dimensional thermal runaway heat generation model and a three-dimensional thermal runaway heat conduction model; and
the three-dimensional thermal runaway heat generation model is established by calculating heat generated by the battery during a thermal runaway process based on the differential scanning calorimeter (DSC) experimental heat of the lithium-ion battery; in a thermal runaway experiment, as the temperature rises, the electrolyte of the lithium-ion battery can react with the anode, further the separator is melted, the cathode and the anode are in direct contact, and then the cathode and the anode can react with each other; as the temperature further rises, the cathode is decomposed and releases heat; and heat transfer is performed inside the battery by means of heat conduction, and convection heat transfer and heat radiation heat transfer are performed between a surface of the battery and an environment.

4. The three-dimensional modeling method for thermal runaway of a lithium-ion battery under different state of charge conditions based on a differential scanning calorimeter experiment according to claim 1, characterized in that in the step S5, the established model is verified through an experimental method, and the method specifically comprises the following steps:
S51: placing the lithium-ion battery into an adiabatic accelerating rate calorimeter to perform the thermal runaway experiment; and
S52: measuring a temperature change of the lithium-ion battery during the thermal runaway process by using a thermocouple, and comparing a measured result with a model result.

5. The three-dimensional modeling method for thermal runaway of a lithium-ion battery under different state of charge conditions based on a differential scanning calorimeter experiment according to claim 1, characterized in that in the step S6, the battery is charged to different states of charge (SOC), namely 100% SOC, 80% SOC, 60% SOC, 40% SOC and 20% SOC respectively, by the charge-discharge meter.
